(19) United States
(12) Patent Application Publication
(10) Pub. No.: US 2005/0208102 A1
(43) Pub. Date: Sep. 22, 2005

(54) Hydrogels used to deliver medicaments to the eye for the treatment of posterior segment diseases

(76) Inventor: Clyde L. Schultz, Ponte Vedra, FL (US)

Correspondence Address: FINCH IP LLC, P.O. BOX 1358, CONCORD, NH, 03302, US

(21) Appl. No.: 10/821,718

(22) Filed: Apr. 9, 2004

Related U.S. Application Data
(60) Provisional application No. 60/461,354, filed on Apr. 9, 2003.

(51) Int. Cl. A61F 2/00; A61K 9/14

(52) U.S. Cl. 424/427; 424/487

(57) ABSTRACT

This invention provides a polymeric drug delivery system including a hydrogel containing one or more drugs for the treatment of a posterior segment disease. Allowing passive transference of this drug from a dilute solution into the hydrogel produces the delivery system. The hydrogel, when placed in contact with the eye, delivers the drug. The delivery of the drug is sustained over an extended period of time, which is of particular utility in the eye, which is periodically flushed with tears. This sustained delivery accelerates the treatment process while avoiding potential damaging effects of localized delivery of high concentrations of compounds, e.g., from eye drops.

### CROSS-REFERENCE TO RELATED APPLICATIONS

This application claims benefit of U.S. Provisional Application No. 60/461,354, filed Apr. 9, 2003, which is hereby incorporated by reference.

### BACKGROUND OF THE INVENTION

In general, the invention relates to the fields of hydrogels, drug delivery systems, and treatment of posterior segment diseases.

Systemic and topical (e.g., via eye drops) administrations of drugs for treatment of diseases of the posterior segment of the eye, such as macular degeneration, are often undesirable. These methods typically require higher total doses of the drug because these routes are inefficient at delivering the drug to the posterior segment. Such high doses increase the cost and may also cause side effects such as local inflammation or adverse systemic reactions. In addition, for most topical treatments, the drug is quickly washed out of the eye, limiting the effective time of treatment.

Thus, sustained-release delivery devices that would continuously administer a drug to the eye for a prolonged period of time are desired for the treatment of posterior segment diseases.

### SUMMARY OF THE INVENTION

The present invention features hydrogel drug delivery systems and methods of producing and using such systems for the treatment of disease in the posterior segment of the eye, e.g., the vitreous, retina (including the macula), choroids, sclera, and optic nerve. The systems are based on a hydrogel into which one or more drugs are passively transferred from a dilute solution, e.g., an aqueous solution. When placed in contact with eye tissue, the drug or drugs passively transfer out of the hydrogel to provide treatment of posterior segment diseases.

Accordingly, in one aspect, the invention features a polymeric hydrogel that contains a drug for the treatment of a posterior segment disease, wherein the drug is capable of being passively released in a therapeutically effective amount to treat the posterior segment disease. Exemplary hydrogel materials include a tetrapolymer of hydroxymethylmethacrylate, ethylene glycol, dimethylmethacrylate, and methacrylic acid. Other examples of hydrogels include etafilcon A, vifilcon A, lidofilcon A, vasurfilcon A, and polymacon B. In addition, variations of these polymers formed by the use of different packing solutions (e.g., phosphate-buffered saline and boric acid) in the manufacturing process are also included. The hydrogel may be ionic or non-ionic. In various embodiments, the drug is capable of being passively released into the ocular environment under ambient or existing conditions. In other embodiments, the hydrogel may be shaped as a contact lens, e.g., one capable of correcting vision. Such a contact lens may be capable of correcting vision in the range of +8.0 to −8.0 diopters or may be plano. The contact lens may also have a base curve between 8.0 and 9.0.

The invention further features a method for making a hydrogel drug delivery system by placing the hydrogel, e.g., a contact lens, in a solution containing one or more drugs as described herein, which is passively transferred to the hydrogel. This method may further include the steps of washing the hydrogel in an isotonic saline solution and partially desiccating the hydrogel prior to placement in the solution. The solution may have, e.g., a pH between 6.9 and 7.4, and a drug concentration of between 0.00001 and 10%. In one embodiment, the hydrogel is placed in the solution of drug for at least 30 minutes.

In another aspect, the invention features a method for treating a posterior segment disease. The method includes placing a hydrogel, as described herein, in contact with an eye, wherein the drug or drugs are passively released from the hydrogel to treat the disease. In various embodiments, the posterior segment disease is in the vitreous, retina (e.g., the macula), choroids, sclera, or optic nerve. The hydrogel may passively release, for example, at least 0.0001, 0.0005, 0.001, 0.005, 0.01, 0.05, 0.1, 0.5, 1, 10, 15, 20, 50, 75, 100, 250, 500, or 1000 μg of a drug, and the hydrogel may be placed in contact with the eye for at least 0.5, 1, 1.5, 2, 2.5, 3, 3.5, 4, 4.5, 5, 7.5, 10, 15, or 24 hours.

Exemplary drugs and posterior segment diseases are described herein.

As used herein, by “ambient conditions” is meant room temperature and pressure.

By “existing conditions” is meant in situ in the eye.

By “treating” is meant medically managing a patient with the intent that a prevention, cure, stabilization, or amelioration of the symptoms will result. This term includes active treatment, that is, treatment directed specifically toward improvement of the disease; palliative treatment, that is, treatment designed for the relief of symptoms rather than the curing of the disease; preventive treatment, that is, treatment directed to prevention of the disease; and supportive treatment, that is, treatment employed to supplement another specific therapy directed toward the improvement of the disease. The term “treating” also includes symptomatic treatment, that is, treatment directed toward constitutional symptoms of the disease.

By “ocular environment” is meant the tissues of and surrounding the eye, including, for example, the sclera, cornea, and other tissues of the ocular cavity and the posterior segment.

The “posterior segment” of the eye includes the vitreous, retina (including the macula), choroids, sclera, and optic nerve.

Exemplary posterior segment diseases include retinal detachment, diabetic retinopathy, macular degeneration (e.g., age-related), proliferative vitreoretinopathy, endophthalmitis, retinopathy of prematurity, posterior segment trauma, intraocular lens-related posterior segment complications, retinal vascular diseases, macular edema, intraocular tumors, hereditary retinal degenerations, AIDS-related retinitis, posterior segment uveitis, and systemic diseases with retinal manifestations. For the purposes of this invention, glaucoma is not a posterior segment disease.

All percentages described in the present invention are by weight unless otherwise specified.

Other features and advantages of the invention will be apparent from the following description and the claims.

### DETAILED DESCRIPTION OF THE INVENTION

This invention provides a polymeric drug delivery system including a hydrogel containing one or more drugs for the treatment of a posterior segment disease. Allowing passive transference of this drug from a dilute solution into the hydrogel produces the delivery system. The hydrogel, when placed in contact with the eye, delivers the drug. The delivery of the drug is sustained over an extended period of time, which is of particular utility in the eye, which is periodically flushed with tears. This sustained delivery may accelerate the treatment process while avoiding potential damaging effects of localized delivery of high concentrations of drugs compared, e.g., to eye drops.

Posterior Segment Diseases

Posterior segment diseases to be treated include, for example, retinal detachment, neovascularization, diabetic retinopathy, macular degeneration (e.g., age-related), proliferative vitreoretinopathy, endophthalmitis, retinopathy of prematurity, posterior segment trauma, intraocular lens-related posterior segment complications, retinal vascular diseases, macular edema (e.g., diabetic), intraocular tumors, retinal degeneration (e.g., hereditary), vascular retinopathy, inflammatory diseases of the retina, AIDS-related retinitis, uveitis, and systemic diseases with retinal manifestations. Neovascularizations include retinal, choroidal, and vitreal. The retinal neovascularization to be treated can be caused by diabetic retinopathy, vein occlusion, sickle cell retinopathy, retinopathy of prematurity, retinal detachment, ocular ischemia, or trauma. The intravitreal neovascularization to be treated can be caused by diabetic retinopathy, vein occlusion, sickle cell retinopathy, retinopathy of prematurity, retinal detachment, ocular ischemia, or trauma. The choroidal neovascularization to be treated can be caused by retinal or subretinal disorders of age-related macular degeneration, diabetic macular edema, presumed ocular histoplasmosis syndrome, myopic degeneration, angioid streaks, or ocular trauma. Other posterior segment diseases are known in the art.

Drug Delivery System

Hydrogels. This invention may employ different polymer compositions. For example, conventional soft contact lenses can be used and can be either ionic or non-ionic hydrogels containing between 10% and 90%, e.g., 24% or 37.5% to 65% or 75%, water by weight and can have any base curve, e.g., from 8.0 to 9.0. The contact lenses may also have the ability to correct vision, for example, over a range of diopters of +8.0 to −8.0. Exemplary hydrogel contact lens materials include etafilcon A, vifilcon A, lidofilcon A, polymacon B, vasurfilcon A, and a tetrapolymer of hydroxymethylmethacrylate, ethylene glycol, dimethylmethacrylate, and methacrylic acid. These materials may also be employed, in other physical forms. Other suitable hydrogel materials are known to those skilled in the art. The hydrogels may be insoluble or may dissolve over time in vivo, e.g., over one day or one week. The drug is passively delivered, for example, by diffusion out of the hydrogel, by desorption from the hydrogel, or by release as the hydrogel dissolves.

The drug delivery system may be produced from a partially desiccated hydrogel (or equivalently a partially hydrated hydrogel). The desiccation step removes, for example, approximately 5%, 10%, 15%, 20%, 25%, 30%, 40%, 50%, 60%, or 75% of the water in the hydrogel. Desiccation can occur, for example, by exposure of the hydrogel to ambient or humidity controlled air, by heating the hydrogel for a specific period of time, or by blowing dried gas, such as N₂, over the hydrogel. In one embodiment, the hydrogel is saturated with physiological (isotonic) saline prior to desiccation. The partially desiccated hydrogel is then soaked, e.g., for at least 30 minutes, in a dilute solution of drug, e.g., at a pH between 6.9 to 7.4. In certain embodiments, the drug is transferred to a contact lens from a non-aqueous solvent, e.g., dimethyl sulfoxide, which may be at least partially removed and replaced with an aqueous solution prior to use in a patient. The hydrogels may also be soaked for at least 1 hour, 6 hours, 12 hours, or 24 hours. The concentration of drug into which the hydrogel is placed is typically 0.000001, 0.000005, 0.00001, 0.00005, 0.0001, 0.0005, 0.001, 0.005, 0.01, 0.05, 0.1, 0.5, 1, 5, 10, 15, 20, 50, 75, 100, 250, 500, or 1000 μg/mL. Higher concentrations may also be used, for example, to reduce the soaking time. The drug is passively transferred into the hydrogel. This transfer may occur at least in part by rehydrating the hydrogel. Diffusion of the drug into the water or polymer in the hydrogel may also occur. In alternative embodiments, a fully hydrated or fully desiccated hydrogel is placed in the soaking solution to produce the medicated hydrogel.

Desirably, the concentration of drug transferred to the hydrogel is substantially lower than the solution in which the hydrogel is soaked. For example, the concentration of growth factor in the hydrogel is at least 2×, 5×, or 10× less than that of the soaking solution. Some drugs, however, may have a higher affinity for a hydrogel than the soaking solution, and such a hydrogel will have a higher concentration of drug than the solution in which it was soaked, e.g., at least 2×, 5×, or 10× more. The water content and type of hydrogel, time and conditions, e.g., temperature of soaking, composition of the soaking solution (e.g., ionic strength and pH), and type of drug employed also may influence the concentration of drug in the drug delivery system. Since the water content of the hydrogel may also help to determine the total amount of drug present in a hydrogel, it represents a variable by which to control the amount of drug delivered to a tissue. The production of a hydrogel containing a specified amount of drug can be accomplished by routine experimentation by one skilled in the art.

Drugs for the Treatment of Posterior Segment Diseases.

Any drug for the treatment of a posterior segment disease may be included in a drug delivery system describe herein. Classes of drugs include anti-infectives (e.g., antibiotics, antibacterial agents, antiviral agents, and antifungal agents); analgesics; anesthetics; antiallergenic agents; mast cell stabilizers; steroidal and non-steroidal anti-inflammatory agents; decongestants; antioxidants; nutritional supplements; angiogenesis inhibitors; antimetabolites; fibrinolytics; neuroprotective drugs; angiostatic steroids; mydriatics; cyclopegic mydriatics; miotics; vasoconstrictors; vasodilators; anticlotting agents; anticancer agents; antisense agents, immunomodulatory agents; carbonic anhydrase inhibitors; integrin antagonists; cyclooxgenase inhibitors; differentiation modulator agents; sympathomimetic agents; VEGF antagonists; immunosuppresant agents; and combinations and prodrugs thereof. Other suitable drugs are known in the art.

Exemplary drugs include 17-ethynylestradiol, 2-ethoxy-6-oxime-estradiol, 2-hydroxyestrone, 2-propenyl-estradiol, 2-propynl-estradiol, 4,9(11)-pregnadien-17α,21-diol-3,20-dione, 4,9(11)-pregnadien-17α,21-diol-3,20-dione-21-acetate, 4-methoxyestradiol, 5-fluorouracil, 6-mannosephosphate, acetazolamide, acetohexamide, acetylcholinesterase inhibitors, acyclovir, adrenal corticalsteroids, adriamycin, aldesleukin, aldose reductase inhbitors, alkylating agents including cyclophosphamide, alpha-tocopherol, amifostine, amphotericin B, anastrozole, anecortave acetate, angiostatic steroids, angiostatin, antazoline, anthracycline antibiotics, antibody to cytokines, anticlotting activase, anti-cytomegalovirus agents, antifibrinogen, antineogenesis proteins, arsenic trioxide, asparaginase, atenolol, atropine sulfate, azacytidine, azathioprine, AZT, bacitracin, bacitracin, betamethasone, betaxolol, bexarotene, bleomycin, busulfan, calcium channel antagonists (e.g., imodipine and diltiazem), capecitabine, carbachol, carmustine, cephalosporin antibiotics, chlorambucil, chloramphenicol, chlorpheniramine, chlorpropamide, chlortetracycline, colchicine, cyclooxgenase II inhibitors, cyclopentolate, cyclophosphamide, cyclosporine, cyclosporine A, cytarabine, cytochalasin B, cytokines, dacarbazine, dactinomycin, daunorubicin, demecarium bromide, dexamethasone, diamox, dichlorphenamide, didanosine, dihydroxylipoic acid, diisopropylfluorophosphate, docetaxel, echinocandin-like lipopeptide antibiotics, echothiophateiodide, eliprodil, endostatin, epinephrine, epirubicin hydrochloride, erythromycin, erythropoietin, eserine salicylate, estradiol, estramustine, etanercept, ethisterone, etoposide, etoposide phosphate, etretinate, eucatropine, exemestrane, famvir, fibrinolysin, filgrastim, floxuridine, fluconazole, fludarabine, fluocinolone, fluoromethalone, fluoroquinolone, fluoxymesterone, flutamide, foscamet, fumagillin analogs, fusidic acid, ganciclovir, gemcitabine HCL, gemtuzumab ozogamicin, gentamicin, glipizide, glutathione, glyburide, goserelin, gramicidin, heat shock proteins, heparin, herbimycon A, homatropine, humanized anti-IL-2receptor mAb (Daclizumab), hydrocortisone, hydroxyamphetamine, hydroxyurea, idoxuridine, ifosfamide, imidazole-based antifungals, insulin, interferon alfa-2a, interferon-gamma, interferons, interleukin-2, irinotecan HCL, ketoconazole, leflunomide, letrozole, leuprolide, levamisole, lidocaine, lipid formulations of antifungals, liposomalamphotericin B, lomustine, macrolide immunosuppressants, matrix metalloproteinase inhibitors, medroxyprogesterone, medrysone, melphalan, memantine, mercaptopurine, mestranol, metals (e.g., cobalt and copper), methapyriline, methazolamide, methotrexate, methylprednisolone, minocycline, mitomycin, mitotane, mitoxantrone hydrochloride, mono and polyclonal antibodies, muramyl dipeptide, mycophenolate mofetil, naphazoline, neomycin, nepafenac, neuroimmunophilin ligands, neurotrophic receptors(Aktkinase), neurotropins, nicotinamide (vitamin B3), nimodipine, nitrofurazone, nitrogen mustard, nitrosoureas, norethynodrel, NOS inhibitors, ondansetron, oprelvekin, oraptamers, oxytetracycline, paclitaxel, pentostatin, pheniramine, phenylephrine, phospholineiodine, pilocarpine, pipobroman, platelet factor 4, platinum coordination complexes (such as cisplatin and carboplatin), plicamycin, polymyxin, prednisolone, prednisone, procarbazine, tacrolimus, prophenpyridamine, prostaglandins, protamine, protease and integrase inhibitors, pyrilamine, rapamycin, ribavirin, rimexolone, rituximab, sargramostim, scopolamine, sodium propionate, streptozocin, succinic acid, sulfacetamide, sulfamethizole, sulfonamides, sulfoxazole, superoxide dismutase, suramine, tamoxifen, temozolomide, teniposide, tetracycline, tetrahydrazoline, thalidomide, thioguanine, thymopentin, thyroid hormones, tolazamide, tolbutamide, topotean hydrochloride, toremifene citrate, transforming factor beta2, trastuzumab, triamcinolone, triazole antifungals, trifluorothymidine, triptorelinpamoate, trisodium phosphonoformate, tropicamide, tumor necrosis factor, uracil mustard, valrubicin, VEGF antagonists (e.g., VEGF antibodies and VEGF antisense), vidarabine, vinblastine, vincristine, vindesine, vitamin B12 analogues, and voriconazole.

A drug may be admixed with a pharmaceutically acceptable carrier adapted to provide sustained release of the drug. Exemplary carriers include emulsions, suspensions, polymeric matrices, microspheres, microcapsules, microparticles, liposomes, lipospheres, hydrogels, salts, and polymers with the drug reversibly bound electrostatically, chemically, or by entrapment. A pharmaceutically acceptable carrier may also include a transscleral diffusion promoting agent, such as dimethylsulfoxide, ethanol, dimethylformamide, propylene glycol, N-methylpyrolidone, oleic acid, isopropyl myristate, polar aprotic solvents, polar protic solvents, steroids, sugars, polymers, small molecules, charged small molecules, lipids, peptides, proteins, and surfactants.

The use of preservatives is non-ideal as they may transfer to a hydrogel at a disproportionately high concentration and cause cytotoxicity.

Treatment Approaches

To treat a posterior segment disease, the hydrogels of the invention are contacted with the ocular fluid of an individual. The hydrogels may be employed in an open or closed eye period. When the system is shaped as a contact lens, the lens may simply be placed in the eye normally in order to deliver the drug. The hydrogel may also be part of a bandage or may be adhered (e.g., by adhesives or sutures) to the eye. If the hydrogel is placed internally in a patient, the hydrogel is advantageously biodegradable. The time period over which the lenses are worn may depend on the level of treatment desired or the amount of drug in the lens. Hydrogels may be considered to be disposable and may be replaced after a specified period of time, e.g., at least 0.5, 1, 1.5, 2, 2.5, 3, 3.5, 4, 4.5, 5, 7.5, 10, 15, or 24 hours. Alternatively, a hydrogel that has a depleted amount of drug may be recycled by soaking the hydrogel again in a solution of drug.

The methods of treatment described herein are capable of delivering a drug to the ocular environment of a patient for a period of time longer than the dwell time achievable by gels or drops. The convenience and simplicity of this system would in many cases enhance patient compliance with therapy.

In certain embodiments, at least 0.001, 0.005, 0.01, 0.05, 0.1, 0.5, 1, 5, 10, 15, 20, 50, 75, 100, 200, 500, 750, or 1000 μg of the drug is released from the hydrogel. This delivery occurs by passive transfer and allows medications to be released into the ocular fluid. The use of hydrogels of the invention may also allow patients to be treated using fewer applications than with traditional methods. In addition, the drug may be released from the hydrogel at a more rapid rate than the release of the drug into a fixed volume of fluid because as the eye produces tears, the drug released is flushed away from the site of application causing an increase in the relative rate of diffusion of the drug out of the hydrogel. The replenishing action of fluids such as tears may also effectively increase the rate of diffusion of the drug into the fluid and lead to earlier onset of therapeutic activity.

In one embodiment, the drug will penetrate the ocular tissue and migrate into the aqueous humor of the eye. Over time, the concentration of the drug will increase such that ocular tissue in the posterior segment of the eye will come into contact with the drug. The drug may have effects on other types of structures, cells, or tissues that may be present at the time of or prior to administration of the drug.

### Other Embodiments

Modifications and variations of the described methods of the invention will be apparent to those skilled in the art without departing from the scope and spirit of the invention. Although the invention has been described in connection with specific desirable embodiments, it should be understood that the invention as claimed should not be unduly limited to such specific embodiments. Indeed, various modifications of the described modes for carrying out the invention, which are obvious to those skilled in the art, are intended to be within the scope of the invention.

Other embodiments are within the claims.

1. A polymeric hydrogel comprising a drug for the treatment of a posterior segment disease, wherein said drug is capable of being passively released in a therapeutically effective amount to treat said posterior segment disease.

2. The polymeric hydrogel of claim 1, wherein said hydrogel has a water content of between 10% and 90%.

3. The polymeric hydrogel of claim 2, wherein said hydrogel has a water content of between 37.5% and 75%.

3. The hydrogel of claim 1, wherein said drug is an anti-infective; analgesic; anesthetic; antiallergenic agent; mast cell stabilizer; steroidal or non-steroidal anti-inflammatory agent; decongestant; antioxidant; nutritional supplement; angiogenesis inhibitor; antimetabolite; fibrinolytic; neuroprotective drug; angiostatic steroid; mydriatic; cyclopegic mydriatic; miotic; vasoconstrictor; vasodilator; anticlotting agent; anticancer agent; antisense agent, immunomodulatory agent; carbonic anhydrase inhibitor; integrin antagonist; cyclooxgenase inhibitor; differentiation modulator agent; sympathomimetic agent; VEGF antagonist; immunosuppresant agent; or combination or prodrug thereof.

4. The hydrogel of claim 1, wherein said drug is selected from the group consisting of 17-ethynylestradiol, 2-ethoxy-6-oxime-estradiol, 2-hydroxyestrone, 2-propenyl-estradiol, 2-propynl-estradiol, 4,9(11)-pregnadien-17α,21-diol-3,20-dione, 4,9(11)-pregnadien-17α,21-diol-3,20-dione-21-acetate, 4-methoxyestradiol, 5-fluorouracil, 6-mannosephosphate, acetazolamide, acetohexamide, acetylcholinesterase inhibitors, acyclovir, adrenal corticalsteroids, adriamycin, aldesleukin, aldose reductase inhbitors, alkylating agents, cyclophosphamide, alpha-tocopherol, amifostine, amphotericin B, anastrozole, anecortave acetate, angiostatic steroids, angiostatin, antazoline, anthracycline antibiotics, antibody to cytokines, anticlotting activase, anti-cytomegalovirus agents, antifibrinogen, antineogenesis proteins, arsenic trioxide, asparaginase, atenolol, atropine sulfate, azacytidine, azathioprine, AZT, bacitracin, bacitracin, betamethasone, betaxolol, bexarotene, bleomycin, busulfan, calcium channel antagonists, imodipine, diltiazem, capecitabine, carbachol, carmustine, cephalosporin antibiotics, chlorambucil, chloramphenicol, chlorpheniramine, chlorpropamide, chlortetracycline, colchicine, cyclooxgenase II inhibitors, cyclopentolate, cyclophosphamide, cyclosporine, cyclosporine A, cytarabine, cytochalasin B, cytokines, dacarbazine, dactinomycin, daunorubicin, demecarium bromide, dexamethasone, diamox, dichlorphenamide, didanosine, dihydroxylipoic acid, diisopropylfluorophosphate, docetaxel, echinocandin-like lipopeptide antibiotics, echothiophateiodide, eliprodil, endostatin, epinephrine, epirubicin hydrochloride, erythromycin, erythropoietin, eserine salicylate, estradiol, estramustine, etanercept, ethisterone, etoposide, etoposide phosphate, etretinate, eucatropine, exemestrane, famvir, fibrinolysin, filgrastim, floxuridine, fluconazole, fludarabine, fluocinolone, fluoromethalone, fluoroquinolone, fluoxymesterone, flutamide, foscamet, fumagillin analogs, fusidic acid, ganciclovir, gemcitabine HCL, gemtuzumab ozogamicin, gentamicin, glipizide, glutathione, glyburide, goserelin, gramicidin, heat shock proteins, heparin, herbimycon A, homatropine, humanized anti-IL-2receptor mAb, hydrocortisone, hydroxyamphetamine, hydroxyurea, idoxuridine, ifosfamide, imidazole-based antifungals, insulin, interferon alfa-2a, interferon-gamma, interferons, interleukin-2, irinotecan HCL, ketoconazole, leflunomide, letrozole, leuprolide, levamisole, lidocaine, lipid formulations of antifungals, liposomalamphotericin B, lomustine, macrolide immunosuppressants, matrix metalloproteinase inhibitors, medroxyprogesterone, medrysone, melphalan, memantine, mercaptopurine, mestranol, metals, cobalt, copper, methapyriline, methazolamide, methotrexate, methylprednisolone, minocycline, mitomycin, mitotane, mitoxantrone hydrochloride, mono and polyclonal antibodies, muramyl dipeptide, mycophenolate mofetil, naphazoline, neomycin, nepafenac, neuroimmunophilin ligands, neurotrophic receptors, neurotropins, nicotinamide, nimodipine, nitrofurazone, nitrogen mustard, nitrosoureas, norethynodrel, NOS inhibitors, ondansetron, oprelvekin, oraptamers, oxytetracycline, paclitaxel, pentostatin, pheniramine, phenylephrine, phospholineiodine, pilocarpine, pipobroman, platelet factor 4, platinum coordination complexes, cisplatin, carboplatin, plicamycin, polymyxin, prednisolone, prednisone, procarbazine, tacrolimus, prophenpyridamine, prostaglandins, protamine, protease and integrase inhibitors, pyrilamine, rapamycin, ribavirin, rimexolone, rituximab, sargramostim, scopolamine, sodium propionate, streptozocin, succinic acid, sulfacetamide, sulfamethizole, sulfonamides, sulfoxazole, superoxide dismutase, suramine, tamoxifen, temozolomide, teniposide, tetracycline, tetrahydrazoline, thalidomide, thioguanine, thymopentin, thyroid hormones, tolazamide, tolbutamide, topotean hydrochloride, toremifene citrate, transforming factor beta2, trastuzumab, triamcinolone, triazole antifungals, trifluorothymidine, triptorelinpamoate, trisodium phosphonoformate, tropicamide, tumor necrosis factor, uracil mustard, valrubicin, VEGF antagonists, VEGF antibodies, VEGF antisense, vidarabine, vinblastine, vincristine, vindesine, vitamin B12 analogues, and voriconazole.

5. The hydrogel of claim 1, wherein said hydrogel comprises a tetrapolymer of hydroxymethylmethacrylate, ethylene glycol, dimethylmethacrylate, and methacrylic acid.

6. The hydrogel of claim 1, wherein said drug is capable of being passively released into an ocular environment under ambient conditions.

7. The hydrogel of claim 1, wherein said drug is capable of being delivered to the posterior segment of the eye.

8. The hydrogel of claim 1, wherein said drug is capable of being delivered to the macula or retina.

9. The hydrogel of claim 1, wherein said drug is capable of being passively released into an ocular environment under existing conditions.

10. The hydrogel of claim 1, wherein said hydrogel is shaped as a contact lens.

11. The hydrogel of claim 10, wherein said hydrogel is capable of correcting vision.

12. The hydrogel of claim 11, wherein said hydrogel is capable of correcting vision in the range of +8.0 to −8.0 diopters.

13. The hydrogel of claim 10, wherein said hydrogel has a base curve between 8.0 and 9.0.

14. The hydrogel of claim 1, wherein said hydrogel comprises an ionic polymer.

15. The hydrogel of claim 1, wherein said hydrogel comprises a non-ionic polymer.

16. The hydrogel of claim 1, wherein said hydrogel comprises etafilcon A, vifilcon A, polymacon B, lidofilcon A, or vasurfilcon A.

17. A method of treating a posterior segment disease, said method comprising contacting an eye of a subject with the hydrogel of claim 1, wherein said hydrogel delivers a therapeutically effective amount of a drug to treat said posterior segment disease.

18. The method of claim 17, wherein said posterior segment disease is selected from the group consisting of retinal detachment, neovascularization, diabetic retinopathy, macular degeneration, proliferative vitreoretinopathy, endophthalmitis, retinopathy of prematurity, posterior segment trauma, intraocular lens-related posterior segment complications, retinal vascular diseases, macular edema, intraocular tumors, retinal degeneration, vascular retinopathy, inflammatory diseases of the retina, AIDS-related retinitis, uveitis, and systemic diseases with retinal manifestations.

19. A method of fabricating a polymeric hydrogel, said method comprising the steps of contacting said polymeric hydrogel with a solution of a drug capable of treating a posterior segment disease, wherein said drug is passively transferred into said hydrogel.
